(19) United States
(12) Patent Application Publication
(10) Pub. No.: US 2012/0123417 A1
(43) Pub. Date: May 17, 2012

(54) Drill Guide with Depth Stop

(75) Inventor: Graham Smith, Newburyport, MA (US)

(73) Assignee: SMITH & NEPHEW, INC., Memphis, TN (US)

(21) Appl. No.: 13/289,659

(22) Filed: Nov. 4, 2011

Related U.S. Application Data
(60) Provisional application No. 61/409,996, filed on Nov. 4, 2010.

(51) Int. Cl. A61B 17/17; A61B 17/16

(52) U.S. Cl. 606/80; 606/98

(57) ABSTRACT

The present invention relates to a drill guide for positioning a bone tunnel in which the drill guide comprises a depth stop. The drill guide comprises an aimer arm and a drill sleeve, and includes a sleeve channel. The drill sleeve is moveable towards and away from the distal end of the aimer arm within the sleeve channel, and the depth stop is adjustable to provide a fixed drilling path length, irrespective of the distance between drill sleeve and distal end of the aimer arm.

The present invention relates to a drill guide for positioning a bone tunnel. In particular, the present invention relates to a drill guide having a depth stop.

When drilling through bone it is desirable to prevent the drill from damaging sensitive tissue by penetrating too far into a joint space or body cavity. A known drill guide for positioning a tibial tunnel requires that once a drilling target has been identified and an aimer arm has been placed on the spot to be drilled, a cannulated drill sleeve is advanced in order to clamp the bony tissue between its tip and the aimer arm tip. The drill sleeve is generally held in place by means of a ratchet mechanism. The bony architecture between the drill sleeve and aimer tips of the drill guide can vary considerably in terms of its width. As a consequence, the distance between the proximal end of the drill sleeve and the tip of the aimer varies. For this reason it is extremely difficult to accurately set a drill to the correct length to avoid over-drilling.

A drill can be physically stopped at its exit point by using a drill guide equipped with an aimer arm having a broad tip, in which the broad tip prevents the drill piece or guidewire from progressing beyond the aimer tip, and thereby prevents overdrilling and tissue damage occurring. This approach requires that the drill is physically stopped at its cutting tip. However, this increases the chance of debris within the joint space caused by the drill or guidewire tip being driven together. If the interaction is severe enough, the drill piece or guidewire may break, or the point or hook it comes into contact with may be worn down.

Alternatively, overdrilling can be prevented by providing a drill guide with a secondary safety stop. The secondary stop helps to create a drilling path of fixed length between the back of the stop and the aimer tip. However, the additional time required to install a secondary safety stop in accessing the guide, for example, in posterior drilling of the glenoid is undesirable.

A more convenient arthroscopic approach could have considerable advantages over existing and open surgical procedures if these obstacles can be overcome. In particular, it could help to prevent the number of instances where damage to tissue caused by overdrilling occurs. Accordingly, there exists a need for a better arthroscopic approach.

The present invention seeks to address at least some of these problems by providing an improved drill guide for drilling a bone tunnel.

In its broadest sense the present invention provides a drill guide for positioning a bone tunnel, the drill guide having an aimer arm and a drill sleeve, in which the drill guide further includes a depth stop to provide a predefined and consistent drilling path length.

According to the present invention there is provided a drill guide for positioning a bone tunnel, the drill guide having an aimer arm and a drill sleeve, wherein the drill guide includes a sleeve channel, and the drill sleeve is moveable within the sleeve channel, towards and away from the distal end of the aimer arm, and wherein the drill guide further includes a depth stop which is adjustable to provide a fixed drilling path length, irrespective of the distance between drill sleeve and distal end of the aimer arm.

Suitably, the depth stop is engageable with the proximal end of the drill sleeve. Preferably, the depth stop is slidably mounted on, or coupled to, a proximal portion of the drill sleeve. Preferably, the depth stop can be advanced distally, or retracted proximally. Suitably, the depth stop comprises a ratchet mechanism. Preferably, the ratchet mechanism includes a pawl and a rack. Preferably, the rack is a series of teeth or radial grooves in the outer surface of the drill sleeve; preferably, a proximal portion of the drill sleeve. According to this arrangement, the pawl is engageable with one or more of the teeth or radial grooves. Suitably, the pawl includes biasing means to urge it towards the teeth or radial grooves of the drill sleeve. Preferably, the biasing means is a spring. Suitably, the pawl is pivotally mounted with the depth stop. In embodiments including this feature, the spring allows the pawl to act as a lever so that it can be disengaged from the teeth or radial grooves at the proximal region of the drill sleeve to allow the drill sleeve to slide freely within the sleeve channel.

Whilst the ratchet mechanism couples the depth stop to the proximal region of the drill sleeve, it may be possible for the depth stop to be inadvertently removed from the proximal end of the drill sleeve. Therefore, in preferred embodiments the drill sleeve and depth stop are further coupled through a slot and pin arrangement. The slot and pin arrangement permits the depth stop to be advanced distally and retracted proximally, relative to the drill sleeve. The pin may be formed with the depth stop, and the slot with the drill sleeve, or the pin formed with the drill sleeve and the slot formed with the depth stop. Suitably, the depth stop includes one or more pins which are slidably engageable within a longitudinal slot in a wall of the drill sleeve. Suitably, the drill sleeve includes a pair of parallel longitudinal opposed slots, and the one or more pins of the depth stop pass through the opposed slots, and are slidably moveable therein. Alternatively, the depth stop includes one or more internal longitudinal grooves for slidable engagement with one or more pins of the drill sleeve. Further alternatively, the depth stop includes one or more longitudinal slots which extend through the walls of the depth stop, from an inner bore within which the drill sleeve is slidable to the outer surface of the depth stop, and the one or more pins of the drill sleeve are slidably engaged within the one or more slots. In embodiments having a pair of slots, the slots are preferably opposed.

Preferably, the drill guide includes a handle to which the aimer arm and the drill sleeve may be connected. Suitably, the handle has an in-line grip. Alternatively, the handle has a pistol grip. Preferably, the aimer arm is formed integrally with the handle. Alternatively, the aimer arm is adjustable relative to the handle.

Suitably, the drill guide includes a sleeve channel for receiving the drill sleeve. Preferably, the sleeve channel is formed with the handle.

Suitably, the drill guide includes advancement means for advancing the drill sleeve towards a distal end of the aimer arm. Suitably, the advancement means also permit retraction of the drill sleeve. Suitably, the advancement means is a ratchet mechanism. Suitably, the ratchet mechanism includes a pawl and a rack, where the rack is located in an outer surface of the drill sleeve. Suitably, the pawl can be disengaged from the rack to allow the drill sleeve to slide freely within the sleeve channel. Alternatively, the drill sleeve can be rotationally advanced and retracted through the sleeve channel. In these alternative embodiments, the outer surface of the drill sleeve includes a thread, the sleeve channel includes a thread, or both the drill sleeve and the sleeve channel comprise a thread.

Preferably, the drill guide includes a lock for locking the drill sleeve in a selected position, relative to the aimer arm.

The drill sleeve is preferably configured to allow a guidewire or drill to be directed into a bone surface to locate a tunnel to be drilled. Preferably, the distal tip of the drill sleeve includes bone engaging means. Suitably, the bone engaging means are one or more teeth or spikes which, in use, act to prevent the distal end of the drill sleeve from slipping when engaged with a bone surface. Suitably, the bone engaging means is an angled opening surrounded by teeth.

Suitably, the orientation of the aimer aim to the guide sleeve are fixed relative to each other. Alternatively, an orientation of the aimer arm may be adjusted with respect to the drill sleeve. The aimer arm may have markings for indicating the orientation of the aimer arm with respect to the drill sleeve.

Preferably, the distal tip of the aimer arm includes bone engaging means. Suitably, the bone engaging means is a hook. Alternatively, the bone engaging means is a ball or point. Suitably, the bone engaging means are engageable with a bone surface distally of the tunnel to be drilled. Suitably, the aimer arm provides an offset to a longitudinal axis of the drill sleeve (i.e., the axis of the bone tunnel).

Another general aspect of the invention features a method for positioning a bone tunnel using the guide discussed above. In use, the distal tip of the aimer arm is positioned against a bone surface, and the drill sleeve advanced so that the bone to be drilled is fixed between the distal tip of the aimer arm and the distal tip of the drill sleeve. The depth stop is then advanced to abut the proximal end of the handle to provide a fixed drilling path length, irrespective of the distance between drill sleeve and distal end of the aimer arm. Next, a guidewire is inserted through the drill sleeve and drilled through the bone. The guide is removed from the patient, leaving the guidewire in place to locate the bone tunnel. Finally, a cannulated drill bit is placed over the guidewire for drilling the bone tunnel, and the tunnel is drilled. Among other advantages, the invention provides an efficient and accurate way of positioning a bone tunnel. The provision of the depth stop according to the invention means that the surgeon is not required to measure the distance between the distal tip of the aimer arm and the proximal end of the drill sleeve. Within the constraints of the drill guide, no matter what the distance is between the distal tips of the aimer arm and the drill sleeve, the distance between the distal tip of the aimer arm and the proximal end of the drill sleeve, and thus the extent to which the drill will be advanced will always be the same. Therefore, the surgeon is able to drill confident in the knowledge that they can drill without the risk of overdrilling, and will not cause any unnecessary damage to surrounding tissues.

A drill guide according to the invention helps to eliminate the requirement for secondary stops, the need to assemble a secondary stop onto the guide after the guide has been deployed, and also the requirement for undesirable aimer tip configurations. Stopping the forward progression of the drill by a means external to the joint, before it can make contact with the tip or hook avoids this problem.

The above and other aspects of the invention will now be described with reference to the following drawings in which:

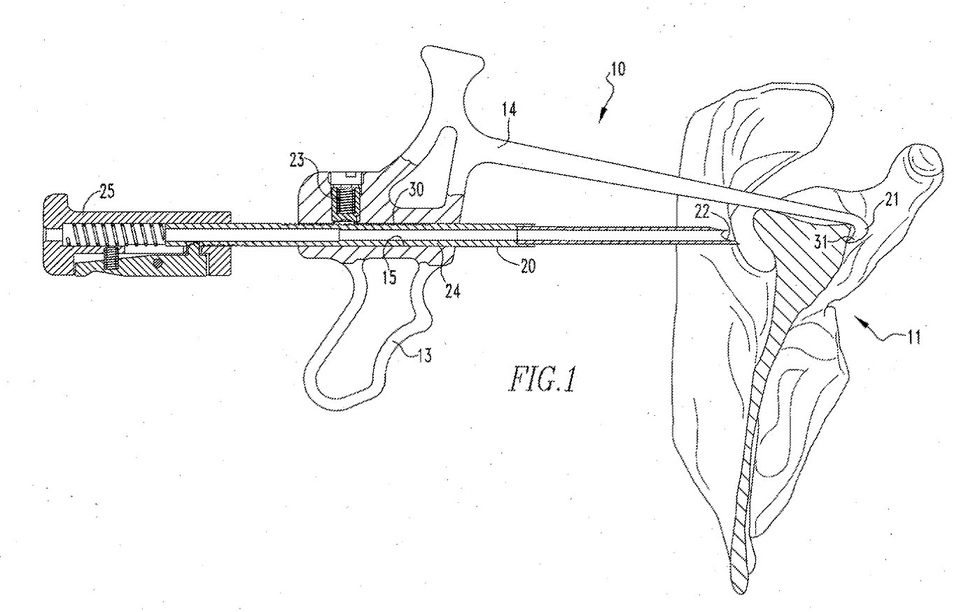

FIG. 1 is a sectional side view of a shoulder joint including an embodiment of an instrument according to the invention, in a partially engaged configuration;

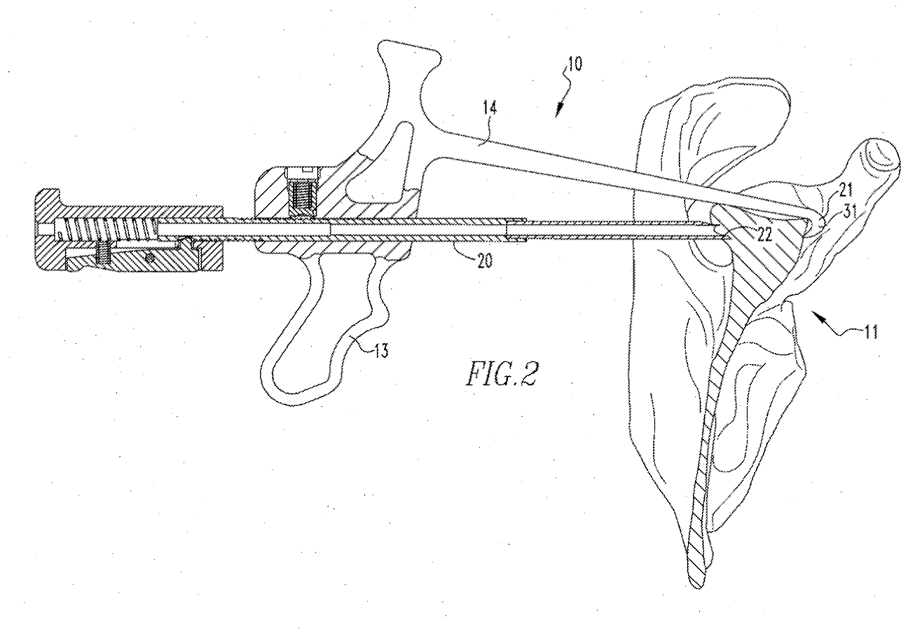

FIG. 2 is a sectional side view of the instrument of FIG. 1 in an intermediate engaged configuration;

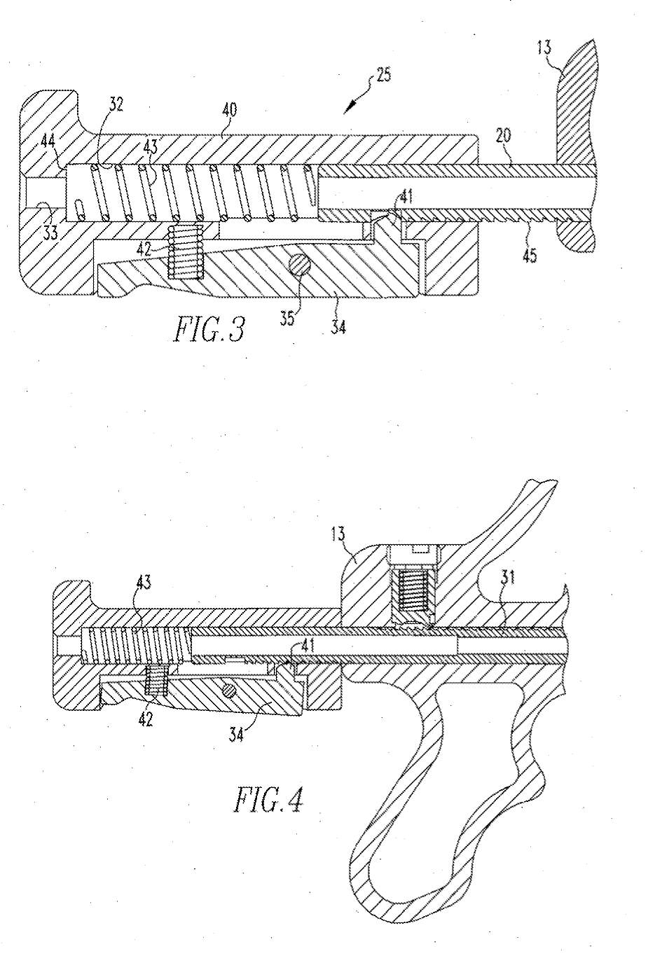

FIG. 3 is a close-up sectional side-view of the proximal end of the instrument of FIG. 1;

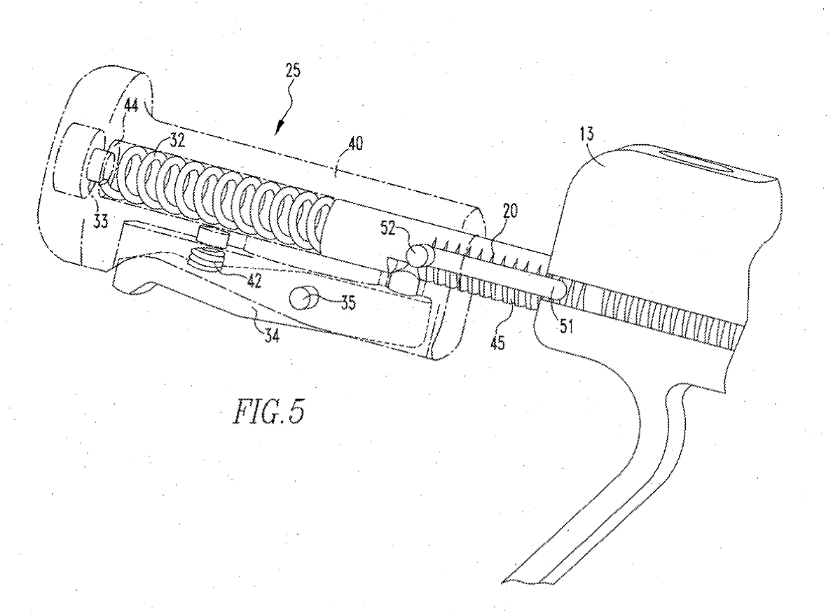

FIG. 4 is a further close-up sectional side-view of the proximal end of the instrument of FIG. 1, in which the instrument is fully engaged;

FIG. 5 is a close-up side-view of the depth stop at the proximal end of the instrument of FIG. 1, in an undeployed position;

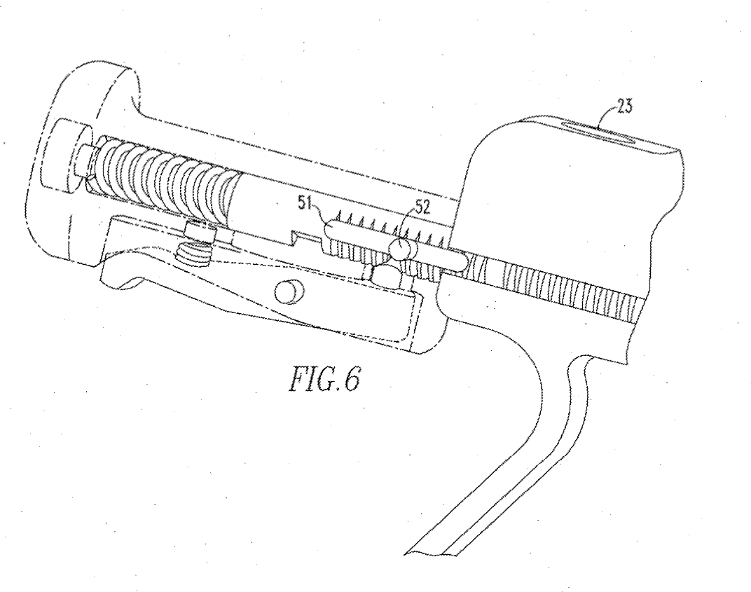

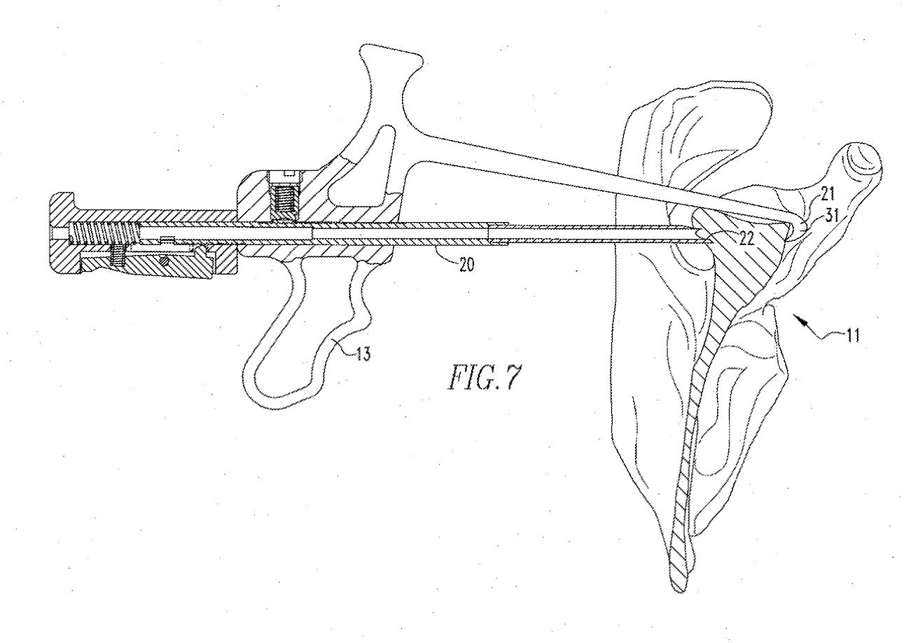

FIG. 6 is a close-up side-view of the depth stop at the proximal end of the instrument of FIG. 1, in a deployed position;

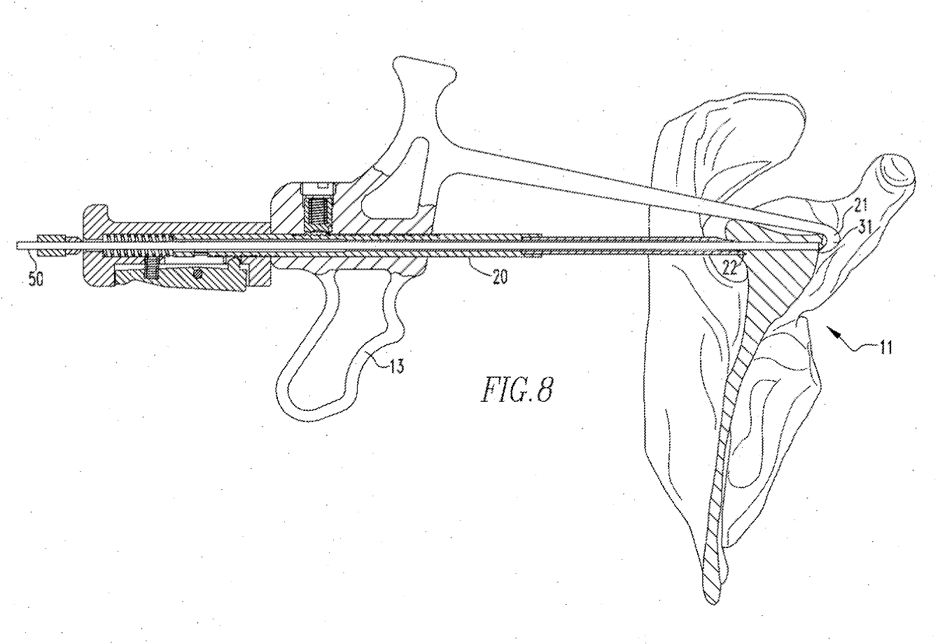

FIG. 7 is full view of the instrument shown in FIG. 4; and

FIG. 8 is a sectional side view of the instrument configuration shown in FIG. 1 in which a drill has been passed through the shoulder joint.

Referring to FIG. 1, there is shown a drill guide 10 for positioning a bone tunnel, and the scapula of a shoulder joint 11, both in section. Drill guide 10 includes a handle 13 located at a first end, in the form of a pistol grip, so that the surgeon may easily grasp and manipulate drill guide 10 during surgery, and an elongate aimer arm 14 towards a second end.

Handle 13 includes a cylindrical sleeve channel 15 for receiving an elongate drill sleeve 20. In the illustrated embodiment, the sleeve channel 15 and drill sleeve 20 are orientated horizontally relative to a slightly backwards sloping handle 13—thus providing a pistol style construction. In alternative embodiments, not shown, the handle is an in-line handle, formed substantially coaxially with the sleeve channel.

In addition, the handle 13 includes a one-way ratchet pawl 23 located adjacent to sleeve channel 15, such that the ratchet pawl 23 can engage with drill sleeve 20 to prevent its movement within sleeve channel 15.

Drill sleeve 20 is sized for insertion through sleeve channel 15, and has an elongated body 24, an angled distal tip 22, and a depth stop in the form of a secondary handle 25 at its proximal end. The elongated body 24 of drill sleeve 20 has a cylindrical bore which provides a passageway for receipt of a guidewire. Drill sleeve 20 also includes a rack 30, in the form of a series of ratchet teeth or radial grooves along one side of body 24. The one-way ratchet pawl 23 of handle 13 engages with the rack 30 and holds the drill sleeve 20 in place within sleeve channel 15. In an alternative embodiment, not shown, the drill can be rotationally advanced or withdrawn through the sleeve channel 15, and the handle includes a moveable pin for engagement with a thread on the outer surface of the drill sleeve. In this alternative embodiment, the drill sleeve includes a proximal portion, a distal portion and a joint between the proximal and distal portions. The joint enables the proximal portion to freely rotate relative to the proximal portion, such that the drill sleeve can be advanced or retracted within the sleeve channel by axial rotation of the proximal end of the drill sleeve. The distal portion of the drill sleeve may have a non-circular cross-section, or may include one of more flanges on guides to prevent axial rotation during advancement or retraction of the drill sleeve so that the orientation of the distal tip is always the same relative to the drill guide.

The depth stop 25, in the form of a secondary handle, at the proximal end of drill sleeve 20 can be used by a surgeon to grasp and manipulate drill sleeve 20 during surgery. Depth stop 25 has a larger outer diameter than that of the sleeve channel 15, such that when drill sleeve 20 is inserted through sleeve channel 15, depth stop 25 prevents drill sleeve 20 from being inserted completely through sleeve channel 15. The depth stop will be discussed in greater detail below, in relation to FIGS. 3 to 6.

As shown, the distal tip 22 of drill sleeve 20 has an angled opening surrounded by teeth. The extent of the angle will largely depend on the architecture of the particular bone surface that is to be drilled.

When drill sleeve 20 is inserted through sleeve channel 15, as shown in FIG. 2, the distal tip 22 provides a stable engagement with a bone surface, here the anterior surface of the scapula, because of the contact made between the teeth and bone surface. Accordingly, the drill sleeve 20 is configured to direct a guidewire into a bone surface to locate the bone tunnel. In addition, drill sleeve 20 supports the guidewire to prevent it from being deflected by the hard bone surface. In the illustrated embodiment, when the distal tip is not angled, there may be a gap between the teeth and the bone surface, which could result in deflection of a guidewire out of the drill sleeve through the gap.

The elongate aimer arm 14 has a proximal arm portion and a distal arm portion. The proximal arm portion is connected to, and extends distally from handle 13. The distal arm portion of the aimer arm 14 includes a distal tip 21 with a spiked hook 31, and is configured to contact a bone surface (e.g., the posterior surface of the scapula). In alternative embodiments, not shown, the tip is pointed or comprises a blunted end, such as a sphericai tip. The distal tip 21 of elongate aimer arm 14 also substantially intersects with a projected path of the drill sleeve 20. In alternative embodiments, not shown, the distal tip of the elongate aimer arm is offset from the projected path of the drill sleeve in further alternative embodiments, not shown, the aimer arm is adjustable relative to the handle to help facilitate access to different surgical sites.

Referring now to FIG. 3, there is shown a depth stop 25 having the form of a handle. The depth stop includes an elongate body 40 which includes a cavity for housing a pawl 34 of a ratchet mechanism. Pawl 34 is mounted about pivot 35 and includes a pin 41 at a first end, and biasing means in the form of a spring 42 at a second end.

The depth stop 25 is cannulated and includes a major bore 32 extending from a distal end of the depth stop 25, and a minor bore 33 which extends from a proximal end. The major and minor bores 32,33 are coaxial and have constant and different diameters, respectively. The diameter of major bore 32 is suitable for receiving the proximal end of drill sleeve 20. However, the drill sleeve 20 cannot enter the minor bore 33 as its diameter is too large to enter the narrower minor bore 33. As a result, the boundary between the two bores prevents further proximal advancement of the drill sleeve. A spring 43 lies within a proximal portion of the major bore 32, adjacent to the minor bore, and abuts against a shoulder 44 where the respective bores meet at one end, and with the distal end of drill sleeve 20 at a second end. In an assembled configuration, in which drill sleeve 20 is inserted into the major bore 32, the spring 43 acts to bias the drill sleeve 20 distally.

In addition, the proximal portion of drill sleeve 20 includes a rack 45 in the form of a series of teeth, to prevent the drill sleeve from being forced out of the depth stop 25 by spring 43. In use, the combination of the pawl 34 and rack 45, allows the depth stop 25 to be advanced incrementally towards the proximal end of handle, or retracted incrementally away from the proximal end of handle as depicted in FIG. 4.

Further, and as shown in FIGS. 5 and 6, to aid advancement and retraction of the depth stop 25 relative to the drill sleeve 20, depth stop includes a longitudinal slot 51 in which a pin 52 at the proximal portion of the drill sleeve 20 can slide. The slot 51 and pin 52 arrangement between the depth stop 25 and the drill sleeve 20 prevents the two components from being separated accidentally, and importantly allows the ratchet 23 in the handle 13 to sit in the groove proximal to the ratchet teeth. This allows the drill sleeve 20 to be advanced distally to capture, in this instance, the glenoid between the hook 31 at the distal end of aimer arm 14, and distal tip 22 of the drill sleeve 20 before the depth stop ratchet 34,45 is activated, to thus account for variations in the width of the glenoid.

The use of drill guide 10 will now be described with reference to the Figures. As noted above, drill guide 10 includes an aimer arm 14. Generally, the aimer arm references a bone surface, such as, for example, the lateral surface of the scapula, for positioning a scapular tunnel. When drill guide 10 is introduced into the patient's shoulder, the surgeon adjusts the orientation of aimer arm 14 until they are satisfied with its positioning, and the spiked hook 31 is embedded in the bone to hold the aimer arm 14 in place (FIG. 1). The drill sleeve 20 is then inserted through sleeve channel 15 of handle 13 so that its angled opening is flush against the angled, anterior surface of the scapula, and its teeth fully contact the bone surface (FIG. 2). The drill sleeve 20 is maintained within the sleeve channel 15 by one-way ratchet 23.

The depth stop is then advanced to abut against the posterior surface of the handle 13 (FIGS. 4 & 5). In doing so a drill path of constant length between the proximal end of the depth stop 25 and the distal tip of drill sleeve 22 is provided, no matter how thick the piece of bone to be drilled is (within the constraints of the instrument). This provides certainty to a surgeon who can be confident that overdrilling will not occur, without the need for taking measurements and calculating tunnel lengths to determine which length of drill piece to use.

Subsequently, and as shown in FIG. 6, a guidewire 50 is then inserted through drill sleeve 20 and drilled through the scapula. As the angled opening is flush against, the angled surface of the scapula, drill sleeve 20 supports the guidewire 50 to the scapula surface to prevent the guidewire from being deflected by the hard bone surface. Drill guide 10 is then removed from the patient's shoulder, not shown, leaving the guidewire 11 in place. Finally, a cannulated drill bit is placed over the guidewire for drilling the bone tunnel (not shown).

1. A drill guide for positioning a bone tunnel, the drill guide comprising an aimer arm and a drill sleeve, wherein the drill guide includes a sleeve channel, and the drill sleeve is moveable towards and away from the distal end of the aimer arm within the sleeve channel, and wherein the drill guide further includes a depth stop which is adjustable to provide a fixed drilling path length, irrespective of the distance between drill sleeve and distal end of the aimer arm.

2. A drill guide according to claim 1, wherein the depth stop is engageable with the proximal end of the drill sleeve.

3. A drill guide according to claim 1, wherein the depth stop is slidably mounted on a proximal portion of the drill sleeve.

4. A drill guide according to claim 1, wherein the depth stop comprises a ratchet mechanism.

5. A drill guide according to claim 1, wherein the drill sleeve and depth stop are further coupled through a slot and pin arrangement.

6. A drill guide according to claim 5, wherein the depth stop comprises one or more longitudinal slots which extend through the walls of the depth stop, and the drill sleeve comprises one or more pins which are slidably engaged within the one or more slots.

7. A drill guide according to claim 1, wherein the drill guide comprises a handle to which the aimer arm and the drill sleeve are connected.

8. A drill guide according to claim 1, wherein the aimer arm is formed integrally with the handle.

9. A drill guide according to claim 1, wherein the drill guide comprises a sleeve channel for receiving the drill sleeve.

10. A drill guide according to claim 9, wherein the sleeve channel is formed with the handle.

11. A drill guide according to claim 1, wherein the drill guide comprises advancement means for advancing the drill sleeve towards a distal end of the aimer arm.

12. A drill guide according to claim 11, wherein the advancement means comprises a ratchet mechanism.

13. A drill guide according to claim 11, wherein the drill sleeve is rotationally advanceable through the sleeve channel.

14. A drill guide according to claim 13, wherein the outer surface of the drill sleeve comprises a thread, the sleeve channel comprises a thread, or both the drill sleeve and the sleeve channel comprise a thread.

15. A drill guide according to claim 1, wherein the drill guide includes a lock for locking the drill sleeve in a selected position, relative to the aimer arm.

16. A drill guide according claim 1, wherein the distal tip of the drill sleeve comprises bone engaging means.

17. A drill guide according to claim 16, wherein the bone engaging means comprise one or more teeth or spikes.

18. A drill guide according to claim 1, wherein the orientation of the aimer aim to the guide sleeve are fixed relative to each other.

19. A drill guide according to claim 1, wherein an orientation of the aimer arm is adjustable with respect to the drill sleeve.

20. A drill guide according to claim 19, wherein the aimer arm comprises orientation markings for indicating the orientation of the aimer arm with respect to the drill sleeve.

21. A drill guide according to claim 1, wherein the distal tip of the aimer arm comprises bone engaging means.

22. A drill guide according to claim 21, wherein the bone engaging means is a hook, pointed tip or blunt end.

23. A drill guide according to claim 1, wherein a distal tip of the aimer arm is offset to a longitudinal axis of the drill sleeve.

24. A method for positioning a bone tunnel using a drill guide according to claim 1, comprising the steps of positioning the distal tip of the aimer arm against a bone surface; advancing the drill sleeve so that the bone to be drilled is fixed between the distal tip of the aimer arm and the distal tip of the drill sleeve; and moving the depth stop to abut the proximal end of the handle to provide a fixed drilling path length, irrespective of the distance between drill sleeve and distal end of the aimer arm.

25. The method according to claim 24 further comprising the steps of inserting a guidewire through the drill sleeve; drilling through the bone; removing the drill guide whilst leaving the guidewire in place to locate the bone tunnel; passing a cannulated drill over the guidewire, and drilling a bone tunnel.
